(19) United States
(12) Patent Application Publication
(10) Pub. No.: US 2003/0017168 A1
(43) Pub. Date: Jan. 23, 2003

(54) Antigen delivery

(76) Inventors: Mary Jo Wick, Onsala (SE); Tomas Leanderson, Malmo (SE); Lars Bjorck, Lund (SE)

Correspondence Address: Thomas E. Popovich, Esq., Popovich & Wiles, PA, Suite 1902, IDS Center, 80 South 8th Street, Minneapolis, MN, 55402, US

(21) Appl. No.: 10/165,055

(22) Filed: Jun. 6, 2002

Related U.S. Application Data
(63) Continuation of application No. PCT/GB00/04830, filed on Dec. 15, 2000.

(30) Foreign Application Priority Data
Dec. 17, 1999 (GB) 9929937.2

(51) Int. Cl. A61K 39/00

(52) U.S. Cl. 424/185.1

(57) ABSTRACT

Protein L, an analogue thereof or a fragment of either thereof is used as a carrier for a heterologous antigen for delivery to an individual. Protein L or an analogue thereof or a fragment of either therof may also be used as an adjuvant composition, to enhance the immune response in the individual to an antigen.

### FIELD OF THE INVENTION

The invention relates to a novel antigen delivery composition and in particular to the use of protein-L of peptostreptococci an analogue thereof or a fragment of either thereof, to deliver an antigen in an individual and/or to enhance the immune response to the antigen in the individual.

### BACKGROUND TO THE INVENTION

Protein-L is an immunoglobulin light chain bonding protein expressed on the surface of approximately 10% of peptostreptococcal strains. Protein-L is a multi-domain protein and has repeat domains showing a substantial degree of homology with each other. Some of these repeat domains are capable of binding to the light chain of immunoglobulin. Protein-L has been isolated from various strains of peptostreptococci and has been cloned and studied in detail from two of these strains. Kastem et al, J. Biol. Chem., 1992, 267, 18, 12820-12825 describe the cloning and expression of protein-L from peptostreptococcal strain 312. Murphy et al, Molecular Microbiology, 1994, 12(6), 911-920 describe the cloning and expression of protein-L from peptostreptococcal strain 3316.

Strain 312 protein-L has 5 immunoglobulin binding domains B1, B2, B3, B4 and B5. Strain 3316 protein-L has 4 immunoglobulin binding domains C1, C2, C3 and C4. Each of these domains has the capacity to bind the light domains and in particular the κ-light chains of human immunoglobuins. Protein-L binds all classes namely IgG, IgA, IgD, IgE, IgM. Protein-L also binds to rabbit, porcine, mouse and rat immunoglobulin. Protein-L has the capacity to bind Fab and scFv fragments by virtue of its interaction with the variable region of kappa light chains of immunoglobulin.

Protein-L is described in EP-A-255497. In view of its capacity to bind immunoglobulins, it has been suggested as a possible therapeutic for the treatment of autoimmune disease.

### SUMMARY OF THE INVENTION

It is now been found that protein-L localises to the spleen in vivo. Protein-L or a fragment thereof stimulates proliferation of splenocytes in vitro and induces the expression of imunostimulatory proteins. The present invention provides a vaccine composition using protein-L or a fragment thereof as an antigen delivery vehicle. Protein L or a fragment thereof may also be used as an adjuvant.

In a first aspect, the invention provides the use of protein-L or an analogue thereof or a fragment of either thereof in the manufacture of a composition for administration to an individual, the polypeptide present as a carrier to deliver an antigen to the individual. This invention also provides a method of enhancing an immune response to an antigen comprising administering a vaccine composition comprising the antigen to an individual and administering protein-L or an analogue thereof or a fragment of either thereof to the individual. The invention also relates to vaccine composition comprising protein-L or an analogue thereof or a fragment of either thereof and an antigen in a pharmaceutically acceptable carrier. In particular, the invention provides a fusion protein comprising protein-L or an analogue thereof or a fragment of either thereof and at least one heterologous antigen.

### IN THE FIGURES

FIG. 1: shows the effect of various immunoglobulin binding proteins on splenocyte proliferation in the presence or absence of polymyxin B (PMB).

FIG. 2: is a titration of the quantity of protein-L required to cause splenocyte proliferation in the presence or absence of PMB.

FIG. 3: shows a titration of the ability of PMB to inhibit LPS-induced splenocyte proliferation

FIG. 4: the effect of 48 hours of co-culture of splenocytes from C57BL/6 mice with protein-L B1-B4, B1-B1, B1 in the presence of 10 μg/ml PMB. Histograms show the fluorescence intensity of the indicated surface molecule on gated B220⁺ cells. Prior to adding biotinylated antibody against the indicated surface molecule, Fc receptors were blocked using mAb 2.4 G2. Cells were also incubated with B1-B4, then incubated with 2.4 G2 and streptavidin-PE, i.e. no primary antibody against a surface molecule was included. Lines in the panel labelled control were incubated with the protein-L fragment, anti-Fc receptor mAb 2.4 G2 was added followed by streptavidin-PE.

FIG. 5: shows constructs of protein L and OVA (257-285).

### DETAILED DESCRIPTION OF THE INVENTION

The invention relates to the use of protein L, an analogue thereof or a fragment of either thereof as a carrier for a heterologous antigen for delivery to an individual. The protein L component assists in delivery of the heterologous antigen to the immune system of the individual to generate an immune response in the individual to the antigen. The invention also relates to the use of protein L or an analogue thereof or a fragment of either thereof to enhance the immune response in an individual to an antigen, for example as an adjuvant Preferably, in respect of all aspects of the invention, the protein L or an analogue thereof or a fragment of either thereof targets delivery of the antigen to the splenocytes and may comprise at least one of the immunoglobulin binding domains of protein. In those embodiments where protein L is being used as a carrier for a heterologous antigen, it is preferred that a single immunoglobulin binding domain be provided for delivery of the antigen to the individual. In those aspects of the invention where protein L or an analogue. thereof or a fragment of either thereof is being used to enhance the immune response in an individual to an antigen, for example, as an adjuvant composition, one or more of the immunoglobulin binding domains of protein L may be used.

The invention may also comprise a peptide analogue or a peptide which is present in an analogue of protein L or a fragment thereof and typically comprises the same sequence as protein L or a fragment thereof, or a sequence which is homologous to part or all of protein L. Preferably, an analogue will have the same sequence or will be homologous to an immunoglobulin binding domain of protein L. References herein to protein L for use as a carrier or an adjuvant include the use of a fragment thereof or an analogue thereof.

Protein L or an analogue thereof or a fragment of either thereof for use in accordance with the invention is hereinafter referred to as a protein L polypeptide. References to a protein L polypeptide are not to be taken as restrictive in limiting the invention to full length protein L but are used to describe the analogue or fragments thereof as described above.

A protein L polypeptide is provided for use as a carrier for a heterologous antigen. The protein L polypeptide is linked or coupled by any suitable means to the antigen to assist in delivery of the antigen to the individual to generate a suitable immune response. A protein L polypeptide for use in accordance with the invention can readily be identified by monitoring the immune response which is generated to an antigen which has been delivered using the protein L polypeptide as a carrier and the antigen which has been delivered without using a carrier. A protein L polypeptide for use as a carrier will assist in the delivery of the antigen or may alter the immune response which is generated to the antigen when compared to that which is generated by administration of the antigen alone without the protein L polypeptide carrier. An alteration in the immune response using a protein L polypeptide as a carrier in accordance with the invention may be an improvement in the immune response such as a larger response due to higher antibody titre, an increase in the duration of the immune response, a quicker reaction to the antigen or a change in the immune response which is generated, for example, a change in the type of antibody response or T cell response which is generated to a specific antigen.

Protein L or an analogue thereof or a fragment of either thereof may be provided for use as an adjuvant. In particular, the protein L polypeptide stimulates a larger, faster or longer lasting immune response against an antigen compared to the response which is seen in the absence of the adjuvant of the invention. An analogue or fragment of protein L in accordance with the invention can readily be identified by monitoring the immune response to an antigen in the presence and absence of the polypeptide. An improvement in the immune response in the presence of the polypeptide demonstrates the utility of the polypeptide as an adjuvant in accordance with the invention. The improvement in the immune response may be a larger immune response such as a higher antibody titer, an increase in the duration of the immune response i.e. a raised antibody titer over a longer period of time or may comprise a quicker reaction to the antigen such that an immune response is seen at a shorter time post administration of the antigen or may result in altered isotype profile compared to the administration of antigen alone.

As described above protein L is expressed by about 10% of peptostreptococcal strains and may be isolated from such bacteria. Preferably, the protein L or an analogue thereof or fragment of either thereof for use in accordance with the invention comprises at least one of the immunoglobulin binding domains of protein L. Examples of these domains are B1, B2, B3, B4 and B5 from peptostreptococcal strain 312, described in detail in Kastem et al, J. Biol. Chem. 1992, 267, 18, 12820-12825, or C1, C2, C3 or C4 from peptostreptococcal strain 3316, described in detail in Murphy et al, Molecular Microbiology 1994, 12(6), 911-920.

Other strains of peptostreptococci may also express protein L. Such protein L variants can be isolated following the cloning methods described in Kastern et al and Murphy et al (supra), if necessary using nucleotides sequences disclosed therein as probes. Fragments of such protein L polypeptides or discrete domains thereof which bind to immunoglobulin light chains can then readily be identified. Single domains may be used in accordance with the invention, in particular as a carrier for an antigen. Alternatively, mixtures of or multiples of such immunoglobulin binding domains may be provided for use in accordance with the invention. Alternatively, an analogue or fragment of protein L for use in accordance with the invention may be identified by monitoring the ability to target delivery of an antigen to splenocytes.

A homologous sequence comprising an analogue of protein L is typically at least 50% and preferably at least 60 or 70 percent and more preferably 80, 90 or at least 95, 97 or 99 percent homologous to protein L, for example over a region of at least 20, preferably at least 30, for instance at least 40, 60 or a 100 or more contiguous amino acids. In preferred embodiments, the analogue is an analogue of one or more of the immunoglobulin binding domains of protein L, such as B1-B5 and C1-C4 identified above. Methods of measuring protein homology are well known in the art and it would be understood by those of skill in the art that in the present context homology is calculated on the basis of amino acid identity (sometimes referred to as hard homology). For example, the UWGCG package provides the BESTFIT programme which can be used to calculate homology (Devereux et al et el (1984) Nucleic Acid Research 1287-395). The programme may be used on its default settings to identify proteins of the invention.

An analogue in accordance with the invention typically differs from the original sequence by substitution, insertion or deletion. Generally, from 1, 2, 3 or 4, or more substitutions deletions or insertions are made, for example over a region of at least 10, preferably at least 20, for instance at least 30, 40, 60 or 100 contiguous amino acids in the analogue. Thus, the homologous sequence may differ from the original sequence by at least 2, 5, 10, 20, 30 or more substitutions, deletions or insertions. The substitutions are preferably conservative. These are defined according to the following table. Amino acids in the same block in the second column and preferably in the same line in the third column maybe substituted for each other.

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

The peptide comprising a fragment of protein L or a fragment of the homologous sequence typically has a length of at least 10, 20, 30, 50 or 100 amino acids in length. Preferably the fragment may have the ability to bind or stimulate proliferation of splenocytes.

Additions may be made to the polypeptides of the invention. An extension may be provided at the N-terminus or C-terminus or both of protein-L or fragment or analogue thereof of the invention. The, or each extension may, for example, be from 1 to 10 amino acids in length. Alternatively, the extension may be longer. The peptides of the invention are for use as carriers or adjuvants for administration with heterologous antigen or tumour antigens and other host antigens with the objective of modifying the immune response to those entities. A peptide for use as an adjuvant may be administered at the same time as the heterologous antigens, or may be administered before or after the antigen. The proteins may be administered through the same or a different route of administration as the antigen. The antigen may be a complete protein or a part of a protein containing an epitope. The antigen may be from a virus, procaryote, or a eucaryote, for example from a bacterium, a yeast, a fungus or a eucaryotic parasite. The antigen may be from an extracellular or intracellular protein. More especially, the antigen sequence may be from gram positive or gram negative bacteria such as E. coli, tetanus or viruses such as hepatitis A, B or C virus, human rhino virus such as type 2 or type 14. herpes simplex virus, polio virus type 2 or 3, foot and mouth disease virus, influenza virus, coxsackie virus or Chlamydia trachomatis.

In a further aspect of the invention, protein L or a fragment or analogue thereof is linked or coupled to an antigen. For example it is provided as a fusion protein or conjugate together with the antigen. Where the antigenic component is conjugated to protein L or a fragment or analogue thereof, this attachment maybe at either the C-terminus or the N-terminus or anywhere between the two termini. The antigenic component may be associated through a non-covalent association, for example by means of hydrophobic interactions, hydrogen bonding or electrostatic interaction.

Preferably, a fusion is provided between protein L polypeptide and the antigen. A composite DNA sequence encoding protein L or a fragment/analogue thereof and the antigen can be prepared by techniques well known in the art The fusion protein can thus be expressed recombinantly from such a DNA sequence. Optionally, a linker nucleic acid sequence may be provided between the two coding sequences such that a linker region is present in the expressed fusion protein. Alternatively, the coding sequences of protein-L or a fragment or analogue thereof may be joined directly together in frame.

The fusion protein of the invention, i.e. protein L polypeptide and the antigen may be present in either orientation, i.e. protein L or fragment or analogue thereof, may be C-terminal or N-terminal to the antigen. Where two or more antigens are provided the protein L component maybe either C-terminal or N-terminal to either or both of the antigens and thus maybe located between the two antigens. A linker peptide maybe present between the components. Typically, the linker will be flexible, allowing movement of the protein L with respect to the antigen. Preferably the linker will not inhibit the correct expression or folding of the components. Preferably, the linker will not be toxic or immunogenic.

Typically, the peptide linker comprises amino acids that do not have bulky side groups and therefore do not obstruct the folding of the protein component. Further, it is preferred to use uncharged amino acids in the linker. Preferred amino acids for use in linkers include glycine, serine, alanine and threonine.

The peptide linker maybe of any suitable length which allows correct folding of the two components. The linker may be from 1 to 4 amino acids in length. Alternatively, the linker maybe from 5 to 50 amino acids in length, for example 10 to 30 amino acids or 15 to 20 amino acids in length.

Preferably, the linker consists essentially of one or more glycine residues and one or more serine residues. Such a linker is termed herein a glycine-serine linker. The linker may contain from 1 to 50, typically 5 to 30 or 10 to 20 glycine residues. The linker may contain from 1 to 50, typically 5 to 30 or from 10 to 20 serine residues. As highlighted above, one or more antigenic sequences may be associated with protein L.

Fusion proteins of the invention may be in substantially isolated form. It will be understood that the fusion proteins may be mixed with carriers of diluents which will not interfere with the intended purpose of the fusion protein and still be regarded as substantially isolated. A fusion protein of the invention may also be in a substantially purified form, in which case it will generally comprise protein in the preparation in which more than 90%, e.g. 95%, 98% or 99% by weight of the protein in the preparation is a fusion protein of the invention.

Fusion proteins of the invention may be modified, for example by the addition of histidine residues to assist their identification or purification or by the addition of a signal sequence to promote their secretion from a cell where the fusion protein does not naturally contain such a sequence.

The invention also provides nucleic acid sequences, i.e. DNA and RNA sequences, encoding the fusion protein of the invention. Polynucleotides of the invention may also be polynucleotides which include within them synthetic or modified nucleotides. A number of different types of modification to polynucleotides are known in the art. These include methyl phosphate and phosphorothioate backbones, addition of acridine or polylysine chains at the 3′ and/or 5′ ends of the molecule. For the purposes of the present invention, it is to be understood that the polynucleotides described herein may be modified by any method available in the art. The invention also provides vectors comprising these nucleic acid sequences; cells containing such vectors or nucleic acid sequences; and methods of producing fusion proteins of the invention, comprising expression of the nucleic acid sequence encoding the fusion protein in a cell, and recovering the fusion protein thus obtained.

A person of skill in the art will be able to generate nucleic acid sequences encoding fusion proteins by techniques well known in the art and will also be able to generate vectors comprising those sequences and transform or transfect cells with such vectors in order to achieve expression of the fusion protein.

In one embodiment, the polynucleotide of the invention in a vector is operably linked to a control sequence which is capable of providing for the expression of the coding sequence by the host cell.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Such vectors may be transformed into a suitable host cell as described above to provide for expression of a fusion protein of the invention. The vectors may be for example, plasmid. virus or phage vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo, for example in a method of gene therapy.

A further embodiment of the invention provides host cells transformed or transfected with the vectors for the replication and expression of polynucleotides of the invention. The cells will be chosen to be compatible with the said vector and may for example be bacterial, yeast, insect or mammalian. Promoters and other expression regulation signals may be selected to be compatible with the host cell for which the expression vector is designed. For example, yeast promoters include S. cerevisiae GAL4 and ADH promoters, S. pombe nmtl and adh promoters. Mammalian promoters include the metallothionein promoter which can be induced in response to heavy metals such as cadmium. For use in insect cells, strong baculovirus promoters such as the polyhedron promoter are preferred. For expression in mammalian cells, strong viral promoters such as the SV40 large T antigen promoter, a CMV promoter or an adenovirus promoter may also be used. All these promoters are readily available in the art.

Suitable cells include cells in which the abovementioned vectors may be expressed. These include microbial cells such as bacteria (e.g. E. coli), mammalian cells such as CHO cells, COS7 cells, P388 cells, HepG2 cells, KB cells, EL4 cells or HeLa cells, insect cells or yeast such as Saccharomyces. Baculovirus or vaccinia expression systems may be used.

Vaccine Formulation

Typically, the vaccines are prepared as injectables, either as liquid solutions or suspensions; solid forms suitable for solution in, or suspension in, liquid prior to injection may also be prepared. The preparation may also be emulsified, or the protein encapsulated in liposomes. The adjuvant or active immunogenic ingredient may be mixed with an excipient which is pharmaceutically acceptable and compatible with the active ingredient. Suitable excipients are, for example, water, saline, dextrose, glycerol, ethanol, or the like and combinations thereof. In addition, if desired, the vaccine or adjuvant may contain minor amounts of auxiliary substances such as wetting or emulsifying agents, pH buffering agents, and/or adjuvants which enhance the effectiveness of the vaccine. An adjuvant or addition adjuvant may be provided in addition to the protein-L component.

Examples of adjuvants which may be effective include but are nor limited to: aluminium hydroxide, N-acetyl-muramyl-L-threonyl-D-isoglutamin (thr-MDP), N-acetyl-nor-muramyl-L-alanyl-D-isoglutamine (CGP 11637, referred to as nor-MDP), N-acetylmuramyl-L-alanyl-D-isoglutamnyl-L-alanine-2-(1′-2′-dipalmitoyl-sn-glycero-3-hydroxvphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

The adjuvants or vaccines are conventionally administered parenterally, by injection, for example, subcutaneously intravenously, transdermally or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccarine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. as a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

The polypeptides of the invention may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

Polynucleotides of the invention can be administered directly as a naked nucleic acid construct to achieve expression of a fusion protein of the invention. Polynucleotides of the invention may also be coadministered with polynucleotides encoding for other proteins such as cytokines, to modify the immune response generated. Alternatively, polynucleotides encoding protein-L or an analogue or fragment thereof can be administered directly as an adjuvant formulation. Uptake of naked nucleic acid construct by mammalian cells is enhanced by several known transfection techniques, for example those including the use of transfection agents. Examples of these agents include cationic agents, for example, calcium phosphate and DEAE-dextran and lipofectants, for example, lipofectam and transfectam. Typically, nucleic acid constructs are mixed with a transfection agent to produce a composition.

Alternatively, a polynucleotide may be delivered to the cells by a viral vector.

Alternatively, the vaccine composition or the adjuvant may be administered as an attenuated strain of a pathogen typically attenuated bacterium. Typically, a DNA sequence encoding protein L or an analogue or fragment thereof or a fusion protein of the invention will be cloned into a vector such as a plasmid to transform an attenuated bacterium such that the protein L or fusion protein thereof is expressed in the attenuated bacterium. Alternatively a DNA sequence encoding protein L or an analogue or fragment thereof or a fusion protein of the invention could be expressed on the chromosome of the attenuated bacteria.

The vaccines are administered in a manner compatible with the dosage formulation and in such amount as will be prophylactically effective. The quantity to be administered, which is generally in the range of 100 μg to 100 mg, preferably 200 μg to 10 mg of antigen per dose, depends on the subject to be treated, capacity of the subject's immune system to synthesize antibodies, and the degree of protection desired. Precise amounts of active ingredient required to be administered may depend on the judgement of the practitioner and may be peculiar to each subject.

The vaccine may be given in a singe dose schedule, or in a multiple dose schedule. A multiple dose schedule is one in which a primary course of vaccination may be with 1-10 separate doses, followed by other doses given at subsequent time intervals required to maintain and or reinforce the immune response, for example at 1 to 4 months for a second dose, and if needed, a subsequent dose(s) after several months or years. The dosage regimen will also, at least in part be determined by the need of the individual and be dependent upon the judgement of the practitioner.

The following Examples illustrate the invention.

### EXAMPLE 1

Experiments were performed to monitor the localisation of protein-L B1-B4 in vivo. 1 μg of ¹²⁵I-Protein L B1-B4 was injected i.v. into the tail vein of C57BL/6 mice. Fifteen minutes, 1 hr, 6 hr and 24 hr post immunization, mice were frozen and embedded in cellulose. Whole sections of mice were subsequently prepared, autoradiography was performed and the level of radioactivity in various organs was quantitated. For each time point a parallel mouse was immunised in an identical fashion for blood sample analysis.

At 15 min post immunization, radioactivity was found around the white pulp of the spleen. The level of isotope within the red pulp was low. Analysis of autoradiographic sections of animals at 1 hr post immunization looked similar to those at 15 min post immunization. At 24 hrs post immunization, isotope was primarily found within the white pulp of the spleen. This appeared to be retention of the label rather than accumulation. The CPM in the white pulp was constant between 6 to 24 hours. In other tissues, such as lung, liver, thymus, splenic red pulp, bone marrow and blood, a decrease in the CPM is noted between 6 and 24 hours. Lymph node and spleen white pulp have high ratios of radioactivity (CPM/mg tissue), and isotope was excreted in the urine. No isotope was apparent in the liver and kidney.

Protein-L B1-B4 traffics to the splenic white pulp after i.v. administration to mice and does not accumulate in the blood, liver, lungs, kidney thymus or bone marrow.

### EXAMPLE 2

Preparations of Protein-L B1-B4, B1-B1 or B1 were incubated with splenocytes from mice for 72 hours and pulse cells with ³H-thymidine to determine proliferation of splenocytes. The ³H-thymidine incorporation assays were performed in the presence or absence of 10 μl/ml polymyxin B (PMB) which binds to and inactivates LPS. This allows the level of potential LPS contamination and its effect on ³H-thymidine incorporation to be assessed.

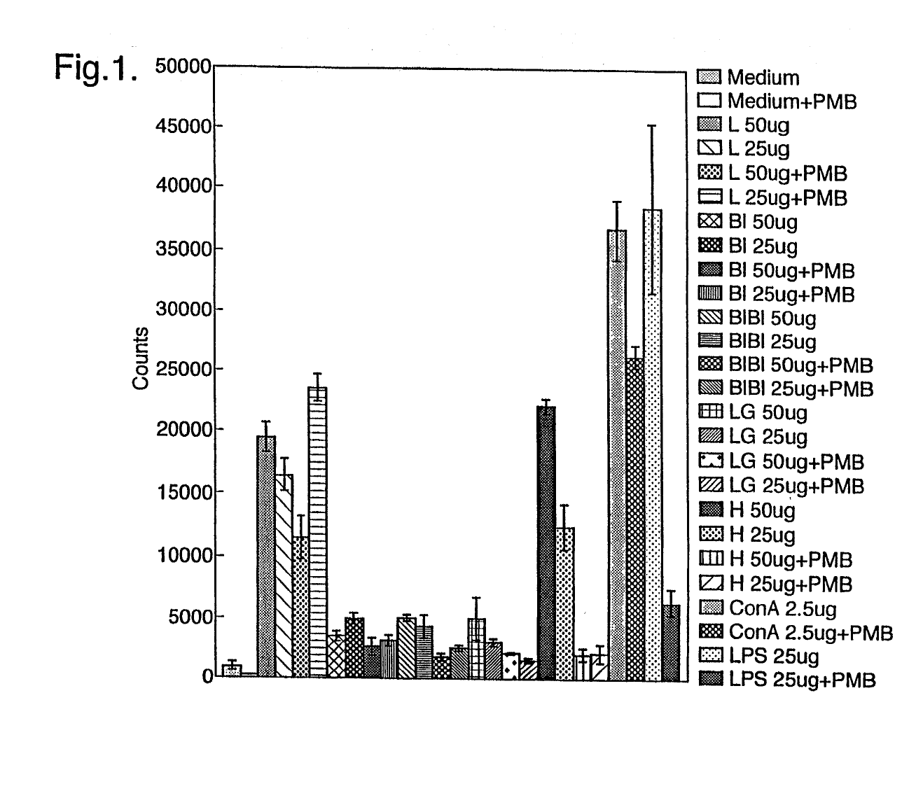

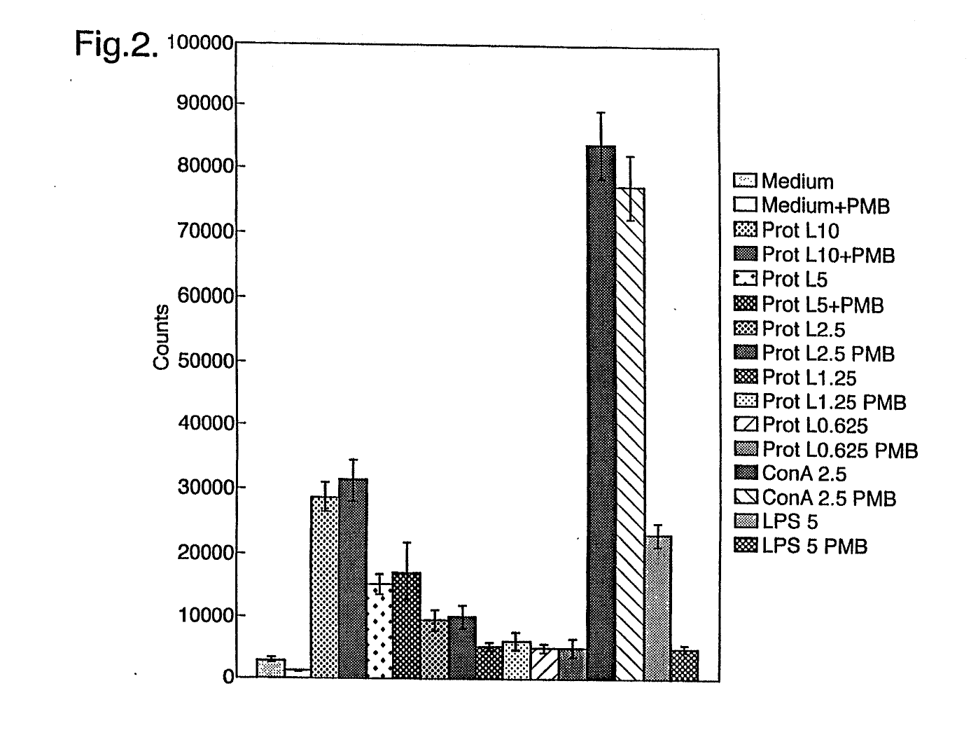

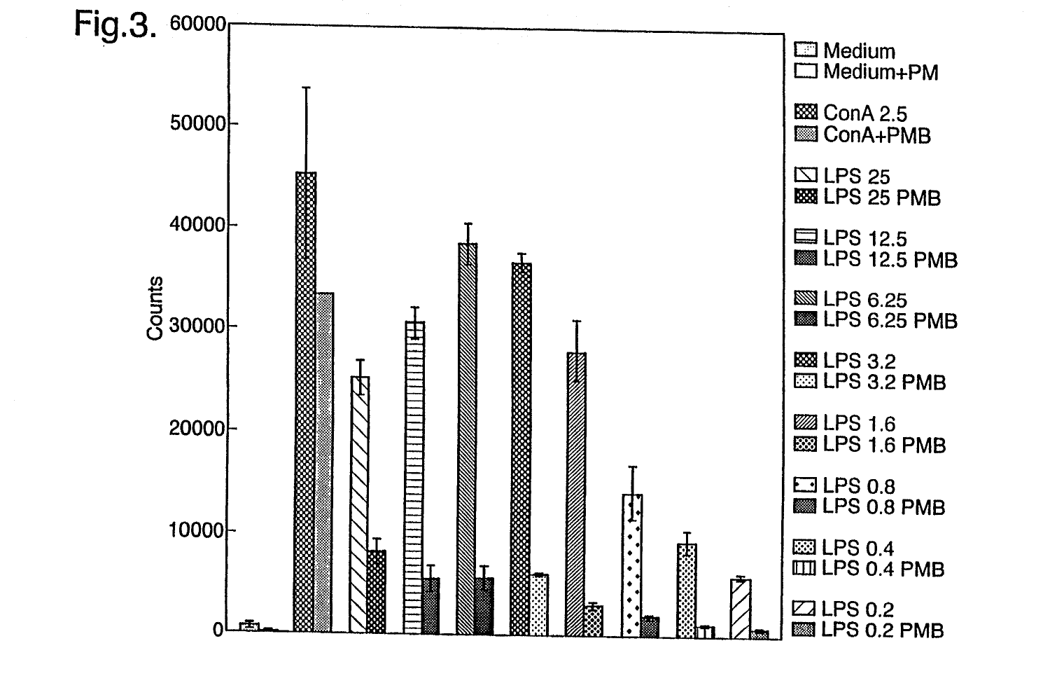

Protein L B1-B4 causes proliferation of murine splenocytes within 72 hrs of culture (FIGS. 1 and 2). This proliferation was not due to LPS contamination as assessed by the lack of effect on ³H-thymidine incorporation in the presence or absence of PMB. Protein L B1-B1 or B1 results in low levels of splenocytes proliferation (FIG. 1). The proliferation observed for the B1 protein is most likely not due to LPS contamination (no effect of PMB), whereas the proliferation resulting from incubation with B1-B1 may be due, at least partially, to LPS (FIG. 1). FIG. 3 shows a titration of the ability of 10 μg/ml PMB to inhibit LPS-induced splenocytes proliferation to demonstrate that a concentration of PMB was used that is capable of inhibiting splenocytes proliferation when <25 μg of purified LPS is added to the culture.

Limulus assays to directly assess LPS contamination in the preparations of Protein L B1-B4, B1-B1 and B1 has shown that the preparation of B1-B4 used in the experiments here was free from LPS while the B1-B1 and B1 preparations had detectable levels of LPS.

Protein-L B1-B4 results in significant proliferation of murine splenocytes whereas Protein-L B1 has a small but reproducible proliferative effect

### EXAMPLE 3

Preparations of Protein L B1-B4, B1-B1 or B1 were incubated with splenocytes from mice for 24, 48 or 72 hours in the presence or absence of 10 μg/ml PMB. The level of surface expression of the costimulatory molecules B7-1, B7-2 and CD40 as well as MHC-I and MHC-II expression on B cells (gated B220⁺ population) was analysed by fluorescence-activated cell sorting (FACS).

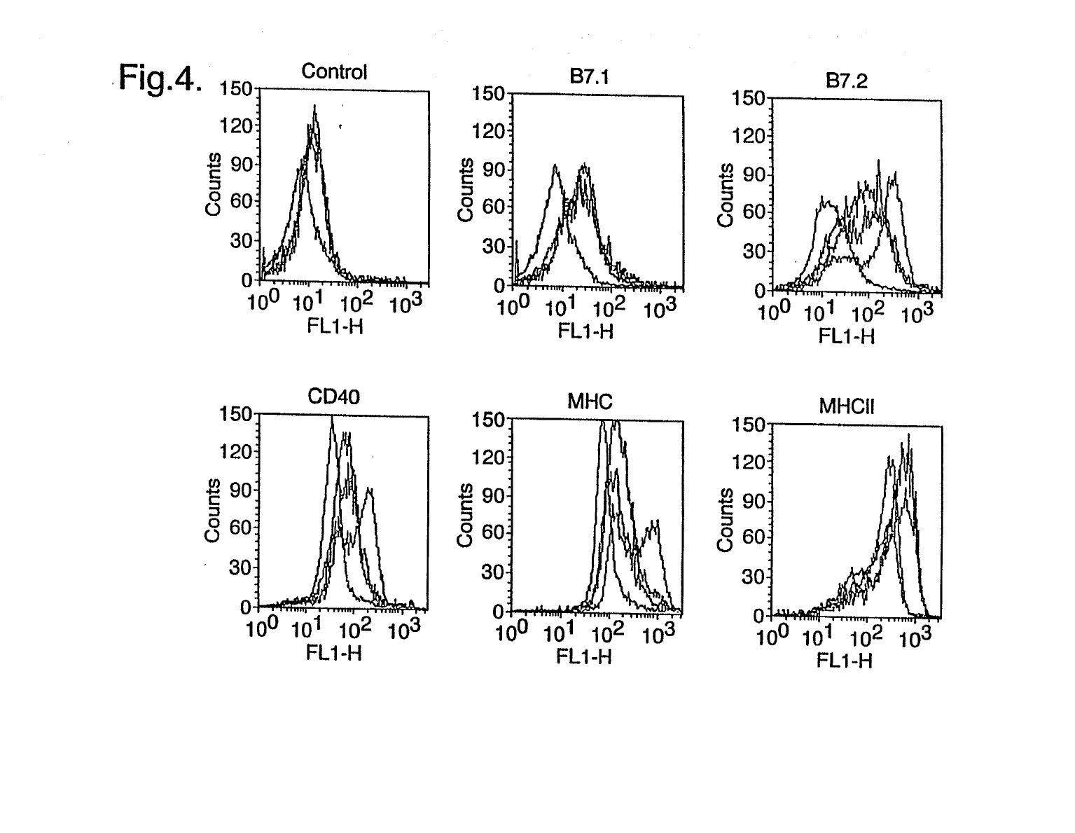

FIG. 4 shows data from a representative FACS experiment after 48 hrs of culture. The data shown in FIG. 4 are from an experiment performed by co-incubating splenocytes with protein L fragments in the presence of 10 μg/ml PMB. Consistent with the proliferation data presented in FIGS. 1-3, no effect of PMB on FACS data was observed for protein-L B1-B4. However, slight alterations in the histograms was observed for co-incubations with B1 and B1-B1 in the presence vs. absence of 10μg/ml PMBB (data not shown).

The most apparent result from these studies is that protein L B1-B4 (5 μg) as well as B1-B1(10 μg) and B1 (10 μg) caused up regulation of B7-2 expression on gated B220⁺ cells, with the most dramatic effect occurring with B1-B4. B1-B4 also upregulated CD40 and MHC-1 expression but had no apparent effect on MHC-II. A slight influence of B1-B1 and B1 on surface expression of CD40 and MHC-I was detectable. The influence of all three protein L fragments on B7-1 expression was small and similar for each of the proteins analysed.

For all protein-L fragments, the effect on surface molecule expression was only slight at 24 hours of co-incubation. The FACS data for 48 hours of incubation was selected to represent the effect of protein L fragments on surface molecule expression of gated B220⁺ splenocytes.

Co-incubation of protein-L B1-B4 with splenocytes results in increased surface expression of B7-2, CD40 and MHC-I on gated B220⁺ cells; a slight alteration in B7-1 expression was evident. Co-incubation of splenocytes with B1-B1 or B1 resulted in increased surface expression of B7-2, but to a lesser degree than that observed with B1-B4. B1-B1 and B1 a slight influence on surface expression of B7-1, CD40, MHC-I and MHC-II expression.

### EXAMPLE 4

Three groups of mice were immunized with either 1)-PBS; 2) 50 μg OVA in PBS; or 3) 50 μg ova mixed with 50 μg protein L B1-B4 in PBS. All mice were immunized on day 0 and boosted 5 weeks later. Immunizations were SC at the base of the tail. Blood samples were taken before immunization and at weeks 2, 4 and 7 during the experiment. Serum from individual mice was tested for anti-OVA IgM, IgG1 and IgG2a antibodies by ELISA.

Data from an in vivo experiment examining the magnitude and isotype of the anti-OVA response are shown in table 1. Table I summarizes the titer of anti-OVA IgG1 for individual mice from this experiment. The titer is defined as the reciprocal of the dilution of sera that gave an OD reading in the ELISA 2.5 times above the background OD for PBS immunized mice. No detectable IgM or IgG2a antibodies recognizing OVA at a titer above PBS immunized was detected f6r any animals.

TABLE 1
Anti OVA IgG1 titers of sera mice immunized as described above.

| | Week post immunization | | |
|---|---|---|---|
| | 2 | 4 | 7 |
| Mouse 7.1, OVA | 0 | 100 | >3200 |
| Mouse 7.2, OVA | 0 | 1600 | >3200 |
| Mouse 7.3, OVA | 0 | 0 | 400 |
| Mouse 8.1, OVA + protein-L | 200 | 0 | Sacrificed |
| | | | for Elispot |
| Mouse 8.2, OVA + protein-L | 200 | 400 | >>>3200 |
| Mouse 8.3, OVA + protein-L | 200 | 1600 | 1600 |

### EXAMPLE 5

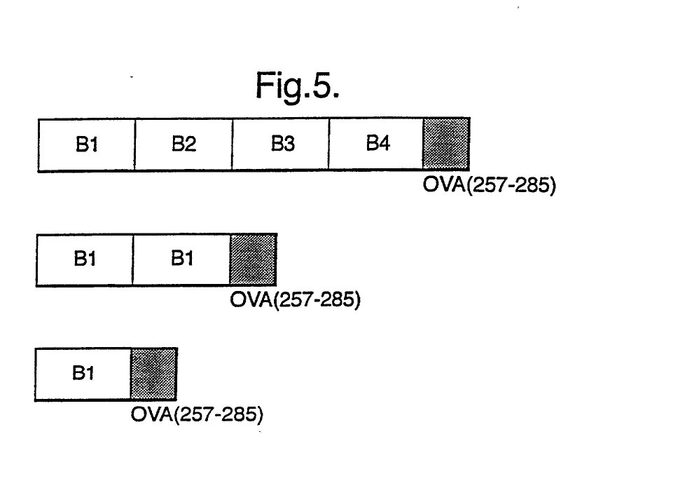

Preparation of constructs of protein L B1-B4 and B 1 with a model antigen containing MIHC-I and MHC-II-binding epitopes fused to the carboxy terminus of protein-L. The following constructs of protein-L have been made: OVA(257-285), which includes the MHC-1-binding (257-264) and the MHC-II-binding (265-280) epitopes from ovalbumin, have been fused to the carboxy terminus of protein-L B1-B4, B1-B1 and B1 as illustrated in FIG. 5. These fusion proteins are used in antigen processing experiments in vitro with nonspecific splenic B cells as antigen presenting cells. The level of antigen presentation can be compared with that obtained using B cells with surface Ig specific for OVA, which is a very efficient antigen presentation system.

These constructs have been confirmed at the DNA level and protein expression has been confirmed by Western blot using antisera against protein-L and OVA. The fusion proteins are in the process of being purified from E. coli for use in in vitro antigen processing experiments and in vivo experiments where the protein L B1-B4-OVA. B1-B1-OVA and BI-OVA fusion proteins are used as immunogens in mice either with or without adjuvant. The immune response is analysed with respect to titer and isotype of anti-OVA antibodies produced, OVA-specific T cell proliferation and cytokine profile of OVA-specific T cells. This is of particular interest in cases when the proteins are administered in PBS without and with adjuvant. The purified proteins will be tested for LPS contamination using the Limulas assay. If LPS is detected, it will be removed using PMB columns.

In the event that the affinity of protein-L for human immunoglobulin is higher than desired and gives suboptimal results upon interaction with human immunoglobulin due to too high affinity, affinity attenuated forms of protein-L can be engineered to specifically lower the affinity for immunoglobulin.

In the event that higher degree of cross-linking of antigen specific immunoglobulin on the surface of B cells is desired, an antigenic construct can be engineered to contain 2 antigenic moieties of the antigen coupled to a protein L component such as a single domain of protein-L (B1) or multiple domain components such as B1-B4 (i.e. a fusion protein containing antigen domain-protein L B1-antigen domain or antigen domain—protein L B1-B4—antigen domain).

To increase the number of B cells targeted, antigenic fusions to protein LA can be made and used. Protein LA contains 4 of the IgGFc- and Fab-binding regions of staphylococcal protein L fused to the carboxyl terminus of 4 of the Igκ light-chain binding domains of protein-L.

1. A vaccine composition comprising protein L or an analogue thereof or a fragment of either thereof coupled to a heterologous antigen and a pharmaceutically acceptable carrier.

2. A vaccine composition according to claim 1 comprising a fission protein of protein L or an analogue thereof or a fragment of either thereof and a heterologous antigen.

3. A vaccine composition according to claim 1 or claim 2 comprising at least one immunoglobulin binding domain of protein L.

4. A vaccine composition according to any one of claims 1, 2 or 3 comprising a single immunoglobulin binding domain of protein L.

5. Use of a polypeptide comprising protein L or an analogue thereof or a fragment of either thereof in the manufacture of a composition for administration to an individual the polypeptide present as an adjuvant to enhance the immune response in the individual to an antigen.

6. Use according to claim 5 wherein the polypeptide comprises at least one of the immunoglobulin binding domains of protein L.

7. Use according to claim 5 or 6 wherein the fragment comprises each of the immunoglobulin binding domain of protein L.

8. Use of a polypeptide according to any one of claims 5 to 7 wherein the polypeptide is formulated together with an antigen for co-administration to an individual.

9. Use of a polypeptide according to claim 8 wherein the vaccine composition comprises a fusion protein of protein L or an analogue thereof or a fragment of either thereof and an antigen.

10. A method of enhancing an immune response to an antigen in an individual comprising administering a vaccine composition comprising the antigen to the individual, and administering protein L an analogue thereof or a fragment of either thereof to the individual.

11. A method according to claim 10 wherein protein L, the analogue thereof or a fragment of either thereof is co-administered with the antigen.

12. A vaccine composition comprising an antigen and protein L, or an analogue thereof or a fragment of either thereof in an amount sufficient to enhance the immune response to the antigen on administration to an individual, and a pharmaceutically acceptable carrier.

13. Use of protein L or an analogue thereof or a fragment of either thereof in the manufacture of a vaccine composition for administration to an individual, the protein L or an analogue thereof or a fragment of either thereof being used as a carrier for a heterologous antigen.

14. Use of a polypeptide according to claim 13 comprising an immunoglobulin binding domain of protein L.
